(19) United States
(12) Patent Application Publication
(10) Pub. No.: US 2025/0072941 A1
(43) Pub. Date: Mar. 6, 2025

(54) Pedicle Screw

(71) Applicant: Chanpin Medtak Co., Ltd., New Taipei City (TW)

(72) Inventors: Fon-Yih Tsuang, Taipei City (TW); Chang Che Yang, Taichung City (TW); Yueh-Feng Chiang, Taichung City (TW); Po-Yi Liu, New Taipei City (TW)

(21) Appl. No.: 18/814,855

(22) Filed: Aug. 26, 2024

(30) Foreign Application Priority Data
Aug. 29, 2023 (TW) 112132620

(51) Int. Cl. A61B 17/72; A61B 17/70

(52) U.S. Cl. CPC A61B 17/725; A61B 17/7091

(57) ABSTRACT

The present invention relates to a pedicle screw including a nail body, a positioning part and a holding part, wherein the positioning part has a first side and a second side facing the first side, in which the nail body has an end disposed on the first side and the holding part is disposed on the second side. The holding part has left and right ends exhibiting a symmetrical arc shape. Thus, when the spinal fixation surgery is conducted, the holding parts of the two adjacent pedicle screws are stacked on each other, and the cross sectional area occupied by the holding parts on the skin surface is reduced, thereby significantly reducing the corresponding opening width, so that the requirement on minimizing the extent of the wound and minimizing the damage of the soft tissue at the surgical site is satisfied.

### CROSS-REFERENCE TO RELATED APPLICATION

This application claims the priority and benefit of Taiwan Patent Application No. 112132620 filed on Aug. 29, 2023 within TIPO, the disclosure of which is incorporated by reference in its entirety as if fully set forth herein.

### BACKGROUND OF THE INVENTION

### 1. Field of the Invention

The present invention relates to a pedicle screw, and more particularly, to a pedicle screw for spinal fixation surgery.

### 2. Description of the Related Art

As a human being grows older, various symptoms may be caused in the body due to aging. In particular, because of standing or sitting for long periods of time at work, it is easy to cause symptoms in which the spine compresses the nerves. When the spine compresses the nerves, medical staffs usually perform minimally invasive surgery on the lumbar spine. Multiple implants are fixed between vertebrae in a specific section to prevent the vertebrae of the section from being displaced, and accordingly, prevent the patient from moving the vertebrae in the section (e.g., prohibit the back from being bent), so that the vertebrae in the section no longer compress the nerves.

When the minimally invasive surgery on the lumbar spine is performed, a minimally invasive wound is required to have a predetermined size since the holding parts connected to adjacent implant ends interfere with each other. Thus, since it is difficult to reduce the area of the minimally invasive wound, subsequent minimally invasive wounds are likely to become inflamed or infected, and it takes a long time to heal the minimally invasive wounds.

In order to solve the above problem, the related art has improved the holding part of the implant to reduce the minimally invasive wounds. For example, according to the intramedullary nail structure described in Taiwan Patent No. TWM566547, a side surface of the holding part connected to the end of the nail body is designed as an inclined plane to overlap inclined surfaces of holding parts with each other and reduce an included angle between the holding parts, so that an opening width of a minimally invasive wound may be reduced.

However, even when the side surface of the holding part is designed as the inclined plane, the opening width of the minimally invasive wound is still wide. Therefore, the holding part is required to be designed for further improvement to dramatically reduce the opening width of minimally invasive wounds, so that the requirement on minimizing the extent of minimally invasive wounds and minimizing the damage of the soft tissue at the surgical site can be satisfied.

### SUMMARY OF THE INVENTION

In view of the above-described defects of the related art, the inventor of the present invention has researched on improvement and innovation and developed a novel pedicle screw for dramatically reducing the opening width of minimally invasive wounds and minimizing the damage of the soft tissue at the surgical site.

An objective of the present invention is to provide a pedicle screw. The pedicle screw includes a nail body, a positioning part and a holding part, wherein the positioning part has a first side and a second side facing each other, in which the nail body has an end disposed on the first side of the positioning part and the holding part is disposed on the second side of the positioning part. In addition, the holding part has left and right ends exhibiting a symmetrical arc shape.

Preferably, the arc shape may have a shape similar to a scimitar sword or a sickle. However, the present invention is not limited thereto. The arc shape may be any shape that allows the neighboring holding parts to be stacked on each other.

The nail body may include a screw shape, and the positioning part may be rotatable with respect to the nail body.

Preferably, the positioning part and the nail body may be rotatable 360 degrees relative to each other.

the positioning part may have an accommodation space, the holding part is formed in both of a front side and a back side thereof with an opening communicating with the accommodation space, and the opening passes through the front side and the back side of the holding part, in which the front side of the holding part is positioned in an inner side of the arc shape and the back side of the holding part is positioned in an outer side of the arc shape.

The accommodation space of the positioning part is provided at the inner top thereof with a screw thread, and the opening of the holding part is provided on the inner bottom thereof with another screw thread. In other words, the screw threads may be provided at connected portions between the second side of the positioning part and the holding part, respectively, and the nail body may be rotated along the two screw threads so as to be placed to the first side of the positioning part.

Preferably, the positioning part and the holding part may have a structure to be integrated with each other, or the positioning part may be assembled with the holding part by a plurality of assembly members. After the pedicle screw is inserted and fixed to the spine in the spinal fixation surgery, and when, for example, the positioning part is configured to be integrated with the holding part, the holding part may be cut or sheared from the positioning part and removed. As another example, when the positioning part and the holding part are assembled by the assembly members, the holding part may be removed from the positioning part by removing the assembly members.

Preferably, the holding part may have a fixed length arrangement or a variable length arrangement. Since patients undergoing minimally invasive lumbar spine surgery have different spinal positions, the fixed length arrangement or the variable length arrangement may be selected depending on the circumstances for the holding part so as to smoothly carry out the surgery.

One other objective of the present invention is to provide a use of the pedicle screw for reducing the opening width of minimally invasive wounds and minimizing the damage of the soft tissue at the surgical site, in which t a front side of a holding part of another pedicle screw may be stacked on the back side of the holding part of the pedicle screw. When the one holding part is stacked on the other holding part, the cross-sectional area of the holding parts may be reduced, thereby reducing the area occupied by the holding parts on a plane, and the included angle between the holding parts may be further reduced compared to the related art. Specifically, by such arrangement, the opening width of the minimally invasive incision may be reduced, and two or more pedicle screws may be placed in the spine through the same opening. Two or more pedicle screws can be stacked as discussed because the spine has certain physiological curvature rather than being straight, and after placing one pedicle screw and adjusting its position such that the narrowest portion of the holding part thereof is at or near the opening of the wound, the one or more subsequent pedicle screws can be placed in the spine through the same opening.

Preferably, with an opening width of the minimally invasive incision having a width of proximately 2 to 3 times of the width of the narrowest portion of the holding part, one or more pedicle screws can be stacked and placed in the spine.

In conclusion, the holding part of the pedicle screw may be designed to have an end in an arc shape to stack on each other. When the spinal fixation surgery is conducted, the holding parts of the two adjacent pedicle screws may be stacked on each other, and the opening area occupied by the holding parts on the skin surface is reduced, such that an opening width of the minimally invasive incision having a width of 2 to 3 times of the width of the narrowest portion of the holding part is required for one or more pedicle screws be placed in the spine. Thus, the corresponding opening width can be significantly reduced, so that the requirement on minimizing the extent of the wound and minimizing the damage of the soft tissue at the surgical site can be satisfied.

Hereinafter, detailed description of specific embodiments will be described with reference to the accompanying drawings, for the further understanding of the object, technical content, features, and effects achieved according to the present invention.

### BRIEF DESCRIPTION OF THE DRAWINGS

FIG. 1 is a perspective view showing a pedicle screw according to the present invention.

FIG. 2 is a left side view showing the pedicle screw according to the present invention.

FIG. 3 is a front view showing the pedicle screw according to the present invention.

FIG. 4 is a schematic view showing a fixed-length form and a variable-length form in a holding part of the pedicle screw according to the present invention.

FIG. 5 is a schematic view in which a holding part of the pedicle screw of the present invention and a holding part of another pedicle screw are stacked on each other.

FIG. 6 is a schematic view showing an included angle in which the holding parts of the pedicle screw according to the present invention are inserted into the spine and then stacked on each other.

### DETAILED DESCRIPTION OF THE INVENTION

The present invention will be described in detail with reference to the accompanying drawings in order to clearly indicate the technical features, contents, advantages and effects to be achieved of the present invention. On the other hand, the drawings used herein are only intended to supplement the specification and may not signify the actual proportions and exact arrangement after the present invention is implemented. Therefore, it is noted that the drawings do not limit the scope of the rights to be actually implemented by the present invention upon interpretation related to the proportions and arrangement of the accompanying drawings.

Hereinafter, in order to fully and clearly disclose the technical content, purpose of the design and the effect to be achieved according to the present invention, the present invention will be described in detail with reference to the disclosed drawings and reference numerals.

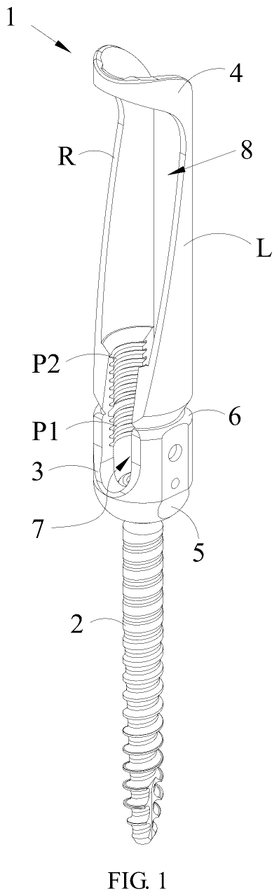

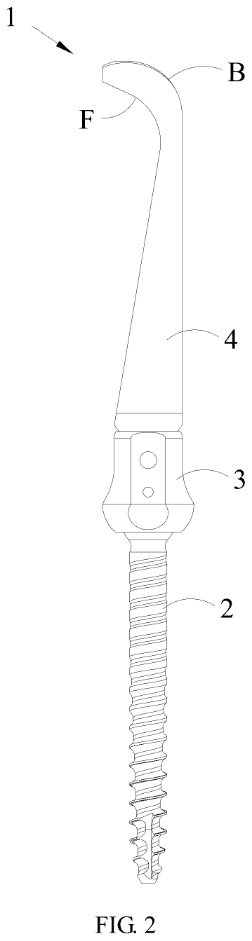

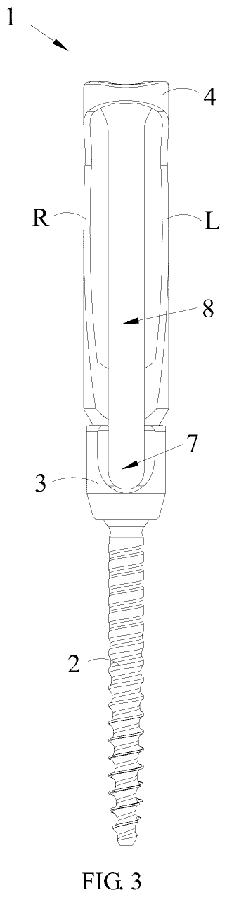

The structure of the pedicle screw of the present invention will now be explained. FIGS. 1, 2 and 3 are respectively a perspective view, a left side view, and a front view of the pedicle screw according to the present invention. As shown in FIGS. 1, 2 and 3, a pedicle screw 1 includes a nail body 2, a positioning

part 3 and a holding part 4, wherein the positioning part 3 has a first side 5 and a second side 6 facing each other, in which the nail body 2 has an end disposed on the first side 5 of the positioning part 3 and the holding part 4 is disposed on the second side 6 of the positioning part 3. In addition, ends of the left L and the right R of the holding part 4 exhibit a symmetrical arc shape.

Preferably, the arc shape may have a shape similar to a scimitar sword or a sickle. However, the present invention is not limited thereto. The arc shape may be any shape that allows the neighboring holding parts to be stacked on each other.

The nail body 2 may have a screw shape and the nail body 2 may be rotatable with respect to the positioning part 3.

Preferably, the positioning part 3 and the nail body 2 may be rotatable 360 degrees relative to each other.

The positioning part 3 may have an accommodation space 7, an opening 8 communicating with the accommodation space 7 may be formed in both of a front side F and a back side B of the holding part 4, and the opening 8 may pass through the front side F and the back side B of the holding part 4, in which the front side F of the holding part 4 is positioned in an inner side of the arc shape and the back side B of the holding part 4 is positioned in an outer side of the arc shape.

The accommodation space 7 of the positioning part 3 may be provided at the inner top thereof with a screw thread P1, and the opening 8 of the holding part 4 may be provided on an inner bottom thereof with another screw thread P2. In other words, the screw thread P1 and the screw thread P2 may be provided at connected portions between the second side 6 of the positioning part 3 and the holding part 4, respectively, and the nail body 2 may be rotated along the two screw threads so as to be placed to the first side 5 of the positioning part 3.

Preferably, the positioning part 3 and the holding part 4 may have a structure to be integrated with each other, or the positioning part 3 may be assembled with the holding part 4 by a plurality of assembly members (not shown). After the pedicle screw 1 is inserted and fixed to the spine to perform spinal fixation surgery, and when, for example, the positioning part 3 is configured to be integrated with the holding part 4, the holding part 4 may be cut or sheared from the positioning part 3 and removed. As another example, when the positioning part 3 and the holding part 4 are assembled by the assembly members, the holding part 4 may be removed from the positioning part 3 by removing the assembly members.

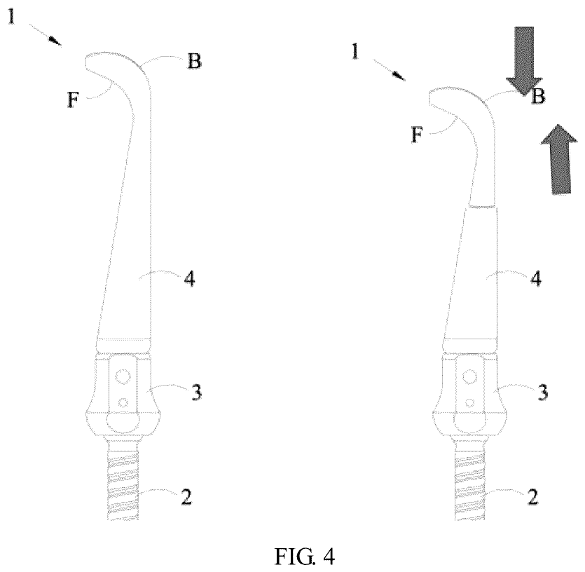

Referring to FIG. 4 together, FIG. 4 is a schematic view showing a fixed-length form and a variable-length form in a holding part of the pedicle screw according to the present invention. Preferably, the holding part 4 may have a fixed length arrangement or a variable length arrangement. Since patients undergoing minimally invasive lumbar spine surgery may have different spinal positions, the fixed length arrangement or the variable length arrangement may be selected depending on the circumstances for the holding part 4 so as to smoothly carry out the surgery.

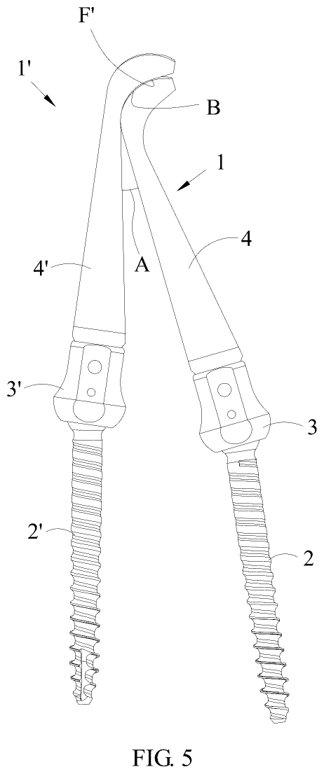

By using the pedicle screw of the present invention, the opening width of minimally invasive wounds may be reduced, and the damage of the soft tissue at the surgical site may be minimized. Referring to FIG. 5, FIG. 5 is a schematic view in which the holding part of the pedicle screw of the present invention and a holding part of another pedicle screw are stacked on each other. As shown in FIG. 5, the back side B of the holding part 4 of the pedicle screw 1 may stack on a front side F′ of another holding part 4′ of another pedicle screw 1′, the cross-sectional area of the holding parts may be reduced, thereby reducing the area occupied by the holding parts on a plane, so that an included angle A between the two holding parts may be further reduced. That is, by such arrangement as discussed, the opening width of the minimally invasive incision may be reduced, and two or more pedicle screws can be stacked under the same opening width, and the use of minimizing the damage of the soft tissue at the surgical site may be realized.

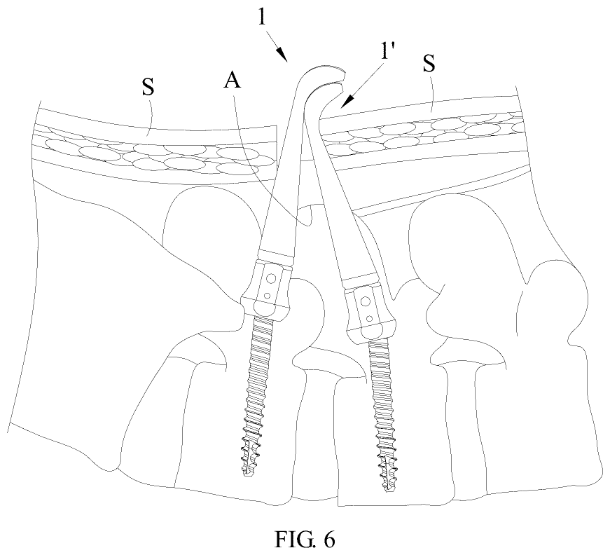

Finally, referring to FIG. 6 together, FIG. 6 is a schematic view showing an included angle in which the holding part of the pedicle screw according to the present invention is inserted into the spine, and then stacked on each other.

As shown in FIG. 6, the holding part 4 of the pedicle screw 1 according to the present invention and the holding part 4′ of the pedicle screw l′ have arc-shaped end portions overlapping with each other. In addition, the narrowest portion of the arch shape of the holding part 4 of the pedicle screw 1′ is positioned at or close to the opening of the wound to maximize the remaining space of the opening, such that subsequent pedicle screw 1 may be placed into the spine with the remaining space of the wound opening. Thus, the holding parts overlap with each other to have an included angle A smaller than that of the pedicle screw in the related art. Specifically, the minimally invasive incision only needs the width of the narrowest arc shape of the holding part 4 to be inserted into the spine; that is to say, only the width of the narrowest arc shape of the holding part 4 is needed during surgery. The size of the minimally invasive incision can be used to place at least one pedicle screw 1 because the spine has a certain physiological curvature rather than being straight. After the first pedicle screw 1′ is placed and the position thereof is adjusted, the second or more pedicle screws 1 may be further placed and stacked with the same opening; preferably, the width of the opening of the minimally invasive incision may be proximately 2 to 3 times of the width of the narrowest portion of the holding part 4, such that one or more pedicle screws 1 as described may be placed in therethrough. In addition, as illustrated in FIG. 6, portions above the narrowest parts of both of the arc-shaped end portion of the holding part 4 and the arc-shaped end portion of the holding part 4′ are exposed above a surface of the skin S.

In conclusion, the pedicle screw according to the present invention is configured to improve the design of the end of the holding part, such that the left and right sides of the end of the holding part exhibit a symmetrical arc shape, so as to further reduce the included angle after overlapping the holding parts with each other. When the spinal fixation surgery is conducted, the holding parts of the two adjacent pedicle screws overlap with each other, and the opening area occupied by the holding parts on the skin surface is reduced, thereby significantly reducing the corresponding opening width, so that the requirement on minimizing the extent of the wound and minimizing the damage of the soft tissue at the surgical site can be satisfied.

The operation, use and effects of the present invention will be fully understood through the description according to the above embodiments. However, the above-described embodiments are only exemplary examples of the present invention, and do not limit the scope for implementing the present invention. Therefore, all simple equivalent modifications and improvements according to the claims and the specification based on the present invention fall within the scope of the present invention.

What is claimed is:

1. A pedicle screw comprising:
a nail body;
a positioning part having a first side and a second side opposite to the first side, the first side being disposed at an end of the nail body; and
a holding part disposed on the second side of the positioning part, the holding part having left and right ends exhibiting a symmetrical arc shape.

2. The pedicle screw of claim 1, wherein the nail body includes a screw shape.

3. The pedicle screw of claim 1, wherein the positioning part is rotatable with respect to the nail body.

4. The pedicle screw of claim 1, wherein the arc shape includes a shape similar to a scimitar sword or a sickle.

5. The pedicle screw of claim 1, wherein the positioning part and the holding part are integrated with each other.

6. The pedicle screw of claim 1, wherein the positioning part and the holding part are assembled with each other through a plurality of assembly members.

7. The pedicle screw of claim 1, wherein the holding part has a fixed length arrangement or a variable length arrangement.

8. The pedicle screw of claim 1, wherein the positioning part includes an accommodation space.

9. The pedicle screw of claim 8, wherein the holding part is formed in both of a front side and a back side thereof with an opening communicating with the accommodation space, and the opening passes through the front side and the back side of the holding part, in which the front side of the holding part is positioned in an inner side of the arc shape and the back side of the holding part is positioned in an outer side of the arc shape.

10. The pedicle screw of claim 9, wherein the back side a front side of a holding part of another pedicle screw is stacked on the back side of the holding part of the pedicle screw.
